(19) United States
(12) Patent Application Publication
(10) Pub. No.: US 2008/0275394 A1
(43) Pub. Date: Nov. 6, 2008

(54) Self-destroy safety syringe

(75) Inventors: Songneng Luo, Puning (CN); Shaoji Luo, Puning (CN)

Correspondence Address: DAVID AND RAYMOND PATENT FIRM, 108 N. YNEZ AVE., SUITE 128, MONTEREY PARK, CA, 91754, US

(73) Assignee: Songneng Luo

(21) Appl. No.: 12/008,490

(22) Filed: Jan. 11, 2008

(30) Foreign Application Priority Data
May 3, 2007 (CN) 200720051269.8
Oct. 25, 2007 (CN) 200710031083.0

(51) Int. Cl. A61M 5/50

(52) U.S. Cl. 604/110

(57) ABSTRACT

A self destroy syringe includes a barrel, a plunger having a hooking ring provided at a top end thereof, and an inner needle seat retaining a needle in position. The inner needle seat is held at a top end of the barrel, wherein the plunger is slid towards the inner needle seat for injection. The inner needle seat has a hooking member provided at a bottom end of the inner needle seat and arranged in such a manner that after injection, the plunger is slidably pushed to engage the hooking member with the hooking ring, such that when the plunger is slidably pulled backward, the inner needle seat and the needle are retracted into the barrel. The structure of the syringe is simple, the requirement of the assembly process is low, and the product pass rate is high. It is suitable for mass product by assembly line.

### BACKGROUND OF THE PRESENT INVENTION

1. Field of Invention

The present invention relates to a syringe, and more particularly to a self destroy safety syringe which retracts the needle into the barrel after it is used.

2. Description of Related Arts

Disposable syringes are widely used in medical area for avoiding cross-infection of diseases in those years. In order to prevent the disposable syringes being reused, self destroying structures are developed for the syringe. Even though the self destroying structure can reduce the chance of cross-infection of diseases owing to its self destroy structure, it still has many drawbacks. The exposed steel needles of the used syringe still cause a lot of trouble during the collecting, transporting, and disposing. Moreover, the people dealing with the used syringes still have the risk of being injured.

The China patent disclosed a needle retractable syringe, wherein the syringe comprises a lock between the bottom of the needle and the plunger. When the medication is injected, the plunger retracts the needle into the barrel. This invention realized self destroy to prevent hurting people, but the needle and the barrel are connected in a tilted manner, the requirement of the assembly process is very high, and the product pass rate is low.

### SUMMARY OF THE PRESENT INVENTION

The major object of the present invention is to provide a needle retractable self destroy safety syringe which is easy to fabricate, has no mediation leaking, and has a high product pass rate.

In order to accomplish the above object, the present invention provides a self destroy safety syringe, wherein the syringe comprises a barrel, a plunger slidably received in the barrel, an outer needle seat mounted at a top end of the barrel, and an elastic piston received in the barrel to engage with a top end of a plunger. An inner needle seat is held by the outer needle seat, wherein a bottom end of the inner needle seat is extended out of the bottom end of the outer needle seat. The inner needle seat comprises a liquid passing channel wherein the needle is held at the liquid passing channel. The inner needle seat comprises a cone shaped hooking member provided at the bottom end thereof. The plunger comprises a hooking ring provided at the top end thereof, wherein the hooking member is arranged to engage with the hooking ring when the hooking member is driven towards the hooking ring. The inner needle seat also comprises a protruding ring outwardly and radially protruded from the outer wall. The outer needle seat 4 also comprises a corresponding holding groove indently and radially on the inner wall, wherein the extended ring is normally engaged with the holding groove.

The outer needle seat can be rotatably threaded onto the barrel. Accordingly, the outer spiral thread is engaged with the inner spiral thread at a position that the bottom end of the outer needle seat is sandwiched between the outer wall of the barrel and the inner sleeve so as to couple the outer needle seat with the barrel.

The elastic piston is made of elastic material adapted to be compressed. The elastic piston comprises a lower ring, a middle ring, and an upper ring. The low ring and the middle ring are in one piece of structure, the upper ring is connected with the middle ring by an elastic compression ring. The elastic piston also comprises a retention groove on the axle through channel. The elastic piston is received in the barrel in this needle retractable self destroy safety syringe at a position that the plunger can reach the “0” calibration without changing the structure of the syringe. The remaining medication in the barrel satisfies the quality requirement.

According to the present invention, the plunger comprises an inner retention groove securely engaging with the top end of the plunger. Cooperating with the elastic piston, the hooking ring can hook up with the hooking member at the bottom of the inner needle seat when the elastic piston is compressed to retract the inner needle seat with the needle together back into the barrel for safe after medication injection. The structure of the present invention is simple, the requirement of the assembly process is low, and the product pass rate is high. It is suitable for mass product by assembly line.

These and other objectives, features, and advantages of the present invention will become apparent from the following detailed description, the accompanying drawings, and the appended claims.

### BRIEF DESCRIPTION OF THE DRAWINGS

FIG. 1 is a sectional view of a self destroy safety syringe of a preferred embodiment of the present invention.

FIG. 2 is a sectional view of the self destroy safety syringe according to the above preferred embodiment of the present invention, illustrating the needle being retracted back to the barrel after the injection.

FIG. 3 is the schematic view of the rubber piston of the self destroy safety syringe according to the above preferred embodiment of the present invention.

FIG. 4 is the partially sectional view of the self destroy safety syringe according to the above preferred embodiment of the present invention.

### DETAILED DESCRIPTION OF THE PREFERRED EMBODIMENT

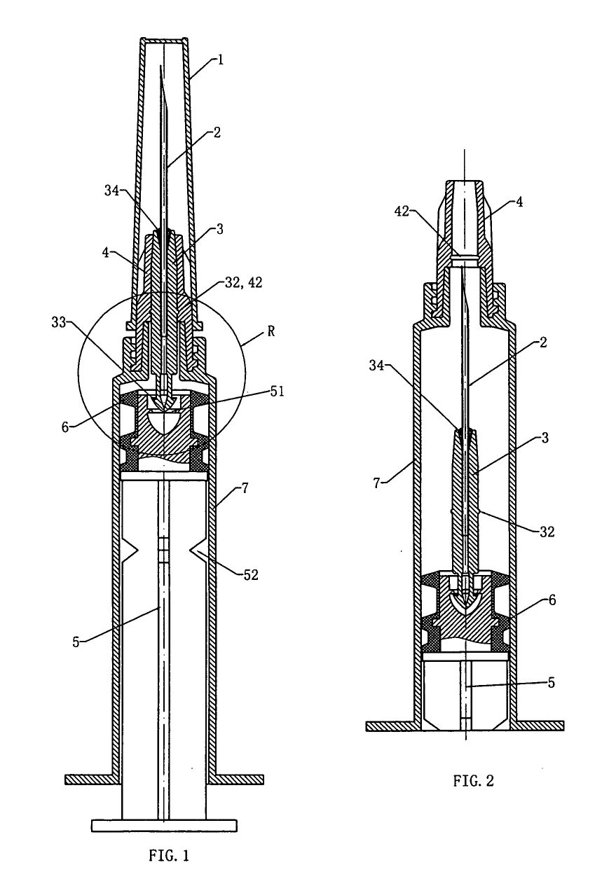

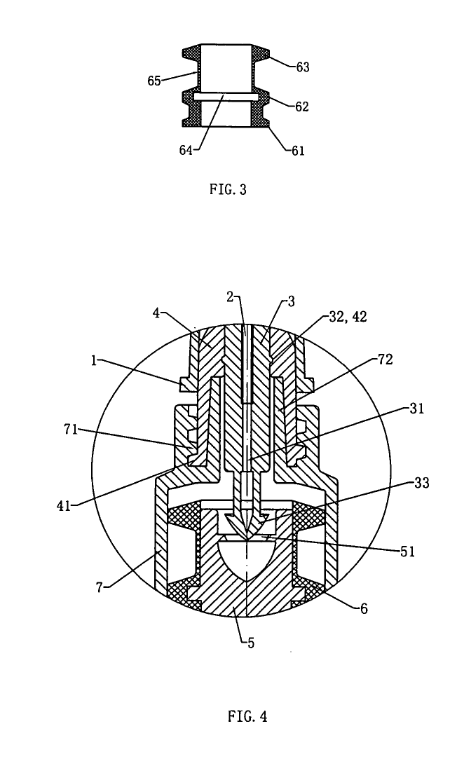

Referring to FIGS. 1 to 4, the needle retractable self destroy safety syringe comprises a barrel 7, an inner needle seat 3, an outer needle seat 4, a plunger 5, and a rubber made elastic piston 6. The plunger 5 is slidably coupled with the barrel 7, wherein a top end of the plunger 5 is coaxially extended through the elastic piston 6. The inner needle seat 3 is coaxially held in the outer needle seat 4, wherein a bottom end of the inner needle seat 3 is extended out of the outer needle seat 4 towards the plunger 5. The inner needle seat 3 comprises a liquid passing channel 31 coaxially formed thereat for the liquid in the barrel 7 passing through the liquid passing channel 31. The needle 2, which is made of stainless steel, is held by the inner needle seat 3 at the liquid passing channel 31. The inner needle seat 3 also comprises a glue chamber 34 at a top opening of the liquid passing channel 31 of the inner needle seat 3. When the needle 2 is positioned inside the inner needle seat 3, a sealing element, such as glue, can be filled inside the glue chamber 34 for fixing the needle 2 so as to retain the needle 2 in position. The inner needle seat 3 comprises a cone shaped hooking member 33 provided at the bottom end thereof. The plunger 5 comprises a hooking ring 51 provided at the top end thereof, wherein the hooking member 33 is arranged to engage with the hooking ring 51 when the hooking member 33 is driven towards the hooking ring 51. Accordingly, the hooking member 33 has a one-directional hooking edge arranged when the hooking member 33 is moved towards the hooking ring 51 at the forward direction, i.e. the pushing direction of the plunger 5, the hooking member 33 is engaged with the hooking ring 51, wherein after the hooking member 33 is engaged with the hooking ring 51, the inner needle seat 3 is locked with the plunger 5. In other words, once the hooking member 33 is engaged with the hooking ring 51, the hooking member 33 cannot be disengaged with the hooking ring 51. The inner needle seat 3 also comprises a protruding ring 32 outwardly and radially protruded from the outer wall. The outer needle seat 4 also comprises a corresponding holding groove 42 indently and radially formed on the inner wall, wherein the protruding ring 32 is normally engaged with the holding groove 42.

In a preferred embodiment, the outer needle seat 4 is rotatably threaded onto the barrel 5. The outer needle seat 4 comprises an outer spiral thread 41 provided at the outer side of the bottom end of the outer needle seat 4. The barrel 5 comprises an inner sleeve 72 and an inner spiral thread 71 at the inner side of the top end of the barrel 5, wherein when the bottom end of the outer needle seat 4 is rotatably coupled with the top end of the barrel 5, the outer spiral thread 41 is engaged with the inner spiral thread 71 at a position that the bottom end of the outer needle seat 4 is sandwiched between the outer wall of the barrel 5 and the inner sleeve 72 so as to retain the outer needle seat 4 at the barrel 5.

In a preferred embodiment, the elastic piston 6, having a hollow structure, is an elastic rubber piston, wherein the elastic piston 6 comprises a lower ring 61, a middle ring 62, and an upper ring 63. The lower ring 61 is integrally and coaxially extended from the middle ring 62 to form a one piece integrated structure, wherein the upper ring 63 is integrally extended from the middle ring 62 through an elastic compression ring 65. In other words, the compression ring 65 is positioned between the middle ring 62 and the upper ring 63, such that when the plunger 5 is pushed to compress the compression ring 65, the hooking member 31 of the inner needle seat 3 is engaged with the hooking ring 51 of the plunger 5. The elastic piston 6 also comprises a retention groove 64 outwardly and radially provided at the inner side of the elastic piston 6 to engage with the top end of the plunger 5.

For using the syringe, the operator must firstly take off the needle cap 1, then suck and inject the medication as normal via the plunger 5. The protruding ring 32 of the inner needle seat 3 and the holding groove 42 of the outer needle seat 4 are engaged together to prevent the inner needle seat 3 from being retracted during sucking medication, and to seal the walls between the inner needle seat 3 and the outer needle seat 4 for preventing the liquid from being leaked. After injection, the plunger 5 is slidably pushed towards the inner needle seat 3 at a position that the hooking member 31 of the inner needle seat 3 is engaged with the hooking ring 51 of the plunger 5. Then, the operator is able to slidably pull back the plunger 5 for retraction. When the pulling force of the plunger 5 is larger than the friction force between the protruding ring 32 and the holding groove 42, the protruding ring 32 will be disengaged with the holding groove 42, such that the inner needle seat 3 is detached from the outer needle seat 4. Therefore, the inner needle seat 3 with the needle 2 together is retracted into the barrel 5. At the end, the operator is able break the plunger 5 at the breakable slit 52 pre-formed thereat. Then, the used needle 2 and liquid medication are kept inside the barrel 7. In other words, the syringe of the present invention is destroyed for safety.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A self destroy syringe, comprising:
a barrel;
a plunger slidably coupled within said barrel, wherein said plunger has a hooking ring provided at a top end thereof; and
an inner needle seat retaining a needle in position, wherein said inner needle seat is held at a top end of said barrel, such that said plunger is slid towards said inner needle seat for injection, wherein said inner needle seat has a hooking member provided at a bottom end of said inner needle seat and arranged in such a manner that after injection, said plunger is slidably pushed to engage said hooking member with said hooking ring, such that when said plunger is slidably pulled backward, said inner needle seat and said needle are retracted into said barrel.

2. The self destroy syringe, as recited in claim 1, further comprising a hollow elastic piston coupling with said top end of said plunger, wherein said elastic piston is compressed when said plunger is pushed to engage said hooking member with said hooking ring.

3. The self destroy syringe, as recited in claim 2, wherein said elastic piston comprises a lower ring, a middle ring integrally and coaxially extended from said lower ring, an upper ring coaxially extended from said middle ring through an elastic compression ring, such that when said plunger is pushed, said compression ring is compressed to allow said hooking member engaging with said hooking ring.

4. The self destroy syringe, as recited in claim 3, wherein said elastic piston further has a retention groove outwardly and radially provided at an inner side of said elastic piston to engage with said top end of said plunger.

5. The self destroy syringe, as recited in claim 1, wherein the hooking member, having a cone shape, has a one-directional hooking edge arranged when said hooking member is engaged with said hooking ring, said inner needle seat is locked up with said plunger.

6. The self destroy syringe, as recited in claim 2, wherein the hooking member, having a cone shape, has a one-directional hooking edge arranged when said hooking member is engaged with said hooking ring, said inner needle seat is locked up with said plunger.

7. The self destroy syringe, as recited in claim 4, wherein the hooking member, having a cone shape, has a one-directional hooking edge arranged when said hooking member is engaged with said hooking ring, said inner needle seat is locked up with said plunger.

8. The self destroy syringe, as recited in claim 1, further comprising an outer needle seat for retaining said inner needle seat in position, wherein said outer needle seat is coupled with said top end of said barrel at a position that said inner needle seat is coaxially held in said outer needle seat, wherein said bottom end of said inner needle seat is extended out of said outer needle seat towards said plunger.

9. The self destroy syringe, as recited in claim 4, further comprising an outer needle seat for retaining said inner needle seat in position, wherein said outer needle seat is coupled with said top end of said barrel at a position that said inner needle seat is coaxially held in said outer needle seat, wherein said bottom end of said inner needle seat is extended out of said outer needle seat towards said plunger.

10. The self destroy syringe, as recited in claim 7, further comprising an outer needle seat for retaining said inner needle seat in position, wherein said outer needle seat is coupled with said top end of said barrel at a position that said inner needle seat is coaxially held in said outer needle seat, wherein said bottom end of said inner needle seat is extended out of said outer needle seat towards said plunger.

11. The self destroy syringe, as recited in claim 8, wherein said inner needle seat comprises a protruding ring outwardly and radially protruded from an outer wall thereof, wherein said outer needle seat has a corresponding holding groove indently and radially formed on an inner wall thereof, wherein said protruding ring is normally engaged with said holding groove to retain said inner needle seat within said outer needle seat, wherein said protruding ring is disengaged with said holding groove when said plunger is pushed to engage said hooking member with said hooking ring.

12. The self destroy syringe, as recited in claim 9, wherein said inner needle seat comprises a protruding ring outwardly and radially protruded from an outer wall thereof, wherein said outer needle seat has a corresponding holding groove indently and radially formed on an inner wall thereof, wherein said protruding ring is normally engaged with said holding groove to retain said inner needle seat within said outer needle seat, wherein said protruding ring is disengaged with said holding groove when said plunger is pushed to engage said hooking member with said hooking ring.

13. The self destroy syringe, as recited in claim 10, wherein said inner needle seat comprises a protruding ring outwardly and radially protruded from an outer wall thereof, wherein said outer needle seat has a corresponding holding groove indently and radially formed on an inner wall thereof, wherein said protruding ring is normally engaged with said holding groove to retain said inner needle seat within said outer needle seat, wherein said protruding ring is disengaged with said holding groove when said plunger is pushed to engage said hooking member with said hooking ring.

14. The self destroy syringe, as recited in claim 10, wherein said outer needle seat comprises an outer spiral thread provided at an outer side of a bottom end of said outer needle seat, wherein said barrel comprises an inner sleeve and an inner spiral thread at an inner side of said top end of said barrel, wherein when said bottom end of said outer needle seat is rotatably coupled with said top end of said barrel, said outer spiral thread is engaged with said inner spiral thread at a position that said bottom end of said outer needle seat is sandwiched between said outer wall of said barrel and said inner sleeve so as to retain said outer needle seat at said barrel.

15. The self destroy syringe, as recited in claim 13, wherein said outer needle seat comprises an outer spiral thread provided at an outer side of a bottom end of said outer needle seat, wherein said barrel comprises an inner sleeve and an inner spiral thread at an inner side of said top end of said barrel, wherein when said bottom end of said outer needle seat is rotatably coupled with said top end of said barrel, said outer spiral thread is engaged with said inner spiral thread at a position that said bottom end of said outer needle seat is sandwiched between said outer wall of said barrel and said inner sleeve so as to retain said outer needle seat at said barrel.

17. The self destroy syringe, as recited in claim 1, wherein said inner needle seat has a liquid passing channel coaxially formed thereat and a glue chamber provided at a top opening of said liquid passing channel of said inner needle seat, wherein said needle is held at said liquid passing channel and sealed by a sealing element at said glue chamber so as to fix said needle at said inner needle seat.

18. The self destroy syringe, as recited in claim 16, wherein said inner needle seat has a liquid passing channel coaxially formed thereat and a glue chamber provided at a top opening of said liquid passing channel of said inner needle seat, wherein said needle is held at said liquid passing channel and sealed by a sealing element at said glue chamber so as to fix said needle at said inner needle seat.

19. The self destroy syringe, as recited in claim 1, wherein said plunger has a breakable slit pre-formed thereat, wherein when said inner needle seat and said needle are retracted into said barrel, said plunger is broken at said breakable slit to keep said inner needle seat and said needle in said barrel.

20. The self destroy syringe, as recited in claim 18, wherein said plunger has a breakable slit pre-formed thereat, wherein when said inner needle seat and said needle are retracted into said barrel, said plunger is broken at said breakable slit to keep said inner needle seat and said needle in said barrel.
